(19) United States
(12) Plant Patent Application Publication
(10) Pub. No.: US 2003/0192095 P1
(43) Pub. Date: Oct. 9, 2003

(54) Chrysanthemum plant named 'Chalunair'

(76) Inventor: Jean-Pierre Challet, Nuaille (FR)

Correspondence Address: BURNS, DOANE, SWECKER & MATHIS, L.L.P., P.O. Box 1404, Alexandria, VA, 22313-1404, US

(21) Appl. No.: 10/114,956

(22) Filed: Apr. 4, 2002

(51) Int. Cl. A01H 5/00

(52) U.S. Cl. PLT/286

(57) ABSTRACT

A new and distinct Chrysanthemum cultivar is provided that is the result of a controlled breeding program. Attractive daisy spoon-type blossoms having red-purple ray florets and green changing to yellow disc florets are formed in profusion. Such blossoms commonly are approximately 6.5 cm in diameter when fully open. The growth habit is low and compact and well-branched and yields a generally uniform and spherical overall plant. The foliage is elegant dark green and contrasts well with the bicolored blossom coloration, and the stems are somewhat flexible. The plant is well suited for growing in pots.

### BOTANICAL/COMMERCIAL CLASSIFICATION

Dendranthema grandifloria/Decorative Pot Mum

### VARIETAL DENOMINATION

cv. ‘Chalunair’

### SUMMARY OF THE INVENTION

The present invention comprises a new and distinct cultivar of Chrysanthemum, botanically known as Dendranthema grandiflora, and hereafter is referred to by the cultivar name ‘Chalunair’.

The new cultivar of the present invention was created at Nuaillé, France during the course of a controlled breeding program. The seeds resulting from the cross were sown and small plants were obtained which were physically and biologically different from each other. Selective study resulted in the identification of a single plant of the new variety of the present invention.

It was found that the new Chrysanthemum cultivar of the present invention displays:

(a) a low compact, well-branched, and generally uniform spherical growth habit,

(b) forms attractive long-lasting decorative daisy spoon-type blossoms having red-purple ray florets and green changing to yellow disc florets,

(c) forms flexible stems,

(d) forms attractive elegant dark green leaves, and

(e) an ability to grow well in pots.

The new cultivar can be grown singly or in clumps in pots. It also can be grown in the landscape. The bicolored red-purple and yellow blossoms contract nicely with the elegant dark green foliage. The blossoms have been found to be long lasting and commonly last approximately three weeks on the plant. Pinching is helpful to further enhance branching; however, such pinching is not necessary since the plant already is naturally self-branching.

Asexual reproduction of the new cultivar by the use of cuttings as performed at Nuaillé, France, in a controlled environment has demonstrated that the characteristics of the new cultivar are firmly fixed and are retained through successive generations of asexual propagation.

‘Chalunair’ has not been observed under all possible environmental conditions to date. Accordingly, it is possible that the phenotype may vary somewhat with variations in the environment, such as temperature, light, day length, contact with pesticides and/or subjection to grown regulation treatments.

### BRIEF DESCRIPTION OF THE PHOTOGRAPH

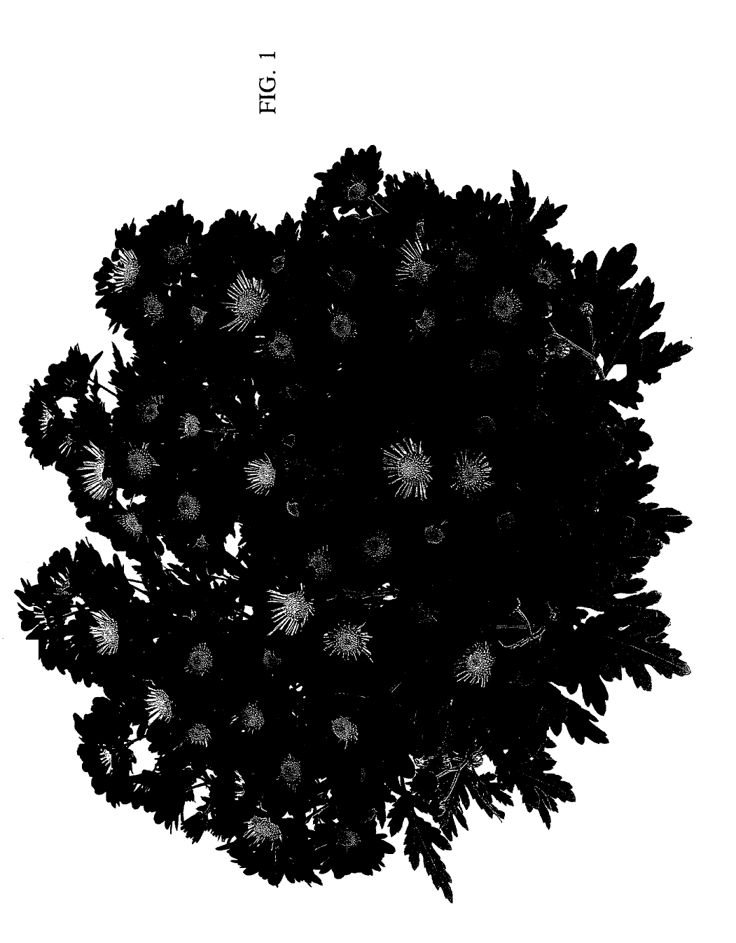

The accompanying FIG. 1 depicts the typical bicolored red-purple and yellow blossoms and elegant foliage of the new ‘Chalunair’ cultivar while growing in a pot in a greenhouse at Nuaillé, France. The ability of the blossom coloration to contrast well with the elegant dark green foliage is illustrated.

### DETAILED DESCRIPTION

The plants described were grown in pots in a greenhouse at Nuaillé, France. No growth regulation was used. The growing conditions approximate those commonly utilized for the commercial production of decorative pot mums.

General Appearance:

Short and stocky with a generally uniform spherical growth habit.

Foliage:

Attractive dark green and elegant.

Stems:

Somewhat flexible.

Flowers:

Possess a daisy spoon-type configuration. The ray florets are red-purple and the disc florets are green changing to yellow with maturity. The diameter is approximately 6.5 cm when fully expanded. The flowering is uniform.

Flower Duration:

Very long lasting with the blossoms commonly lasting approximately three weeks on the plant.

Response Time:

The time between the beginning of the short day period and the flowering date at normal temperature and light intensity is rapid and approximately seven and one-half weeks.

Usage:

Decorative pot mum.

I claim:

1. A new and distinct cultivar of Chrysanthemum plant that displays:
(a) a low compact, well-branched, and generally uniform spherical growth habit,
(b) forms attractive long-lasting decorative daisy spoon-type blossoms having red-purple ray florets and green changing to yellow disc florets,
(c) forms flexible stems,
(d) forms attractive elegant dark green leaves, and
(e) an ability to grow well in pots;
substantially as illustrated and described.
